(19) United States
(12) Patent Application Publication
(10) Pub. No.: US 2026/0207241 A1
(43) Pub. Date: Jul. 23, 2026

(54) ELECTROSURGICAL MEDICAL DEVICE

(71) Applicant: Boston Scientific Medical Device Limited, Galway (IE)

(72) Inventors: Yin Xia, Shanghai (CN); Kensuke Hayashi, Penang (MY)

(73) Assignee: Boston Scientific Medical Device Limited, Galway (IE)

(21) Appl. No.: 19/451,191

(22) Filed: Jan. 16, 2026

(30) Foreign Application Priority Data
Jan. 17, 2025 (CN) 202510084179.1

(51) Int. Cl. A61B 18/00

(52) U.S. Cl. CPC A61B 18/00; A61B 2018/00083; A61B 2018/00178; A61B 2018/00184; A61B 2018/00601; A61B 2018/00744; A61B 2018/0091; A61B 2018/00982; A61B 2218/002

(57) ABSTRACT

A medical device may include an elongate shaft, an end effector, a fluid channel, and a control wire. The end effector may be located at a distal end of the elongate shaft and a fluid channel may extend through the elongate shaft to the end effector. The control wire may extend through the elongate shaft for controlling operation of the end effector. The control wire may be electrically conductive and deliver electrical power to the end effector. The end effector may be configured to selectively output fluid passing through the fluid channel.

### CROSS-REFERENCE TO RELATED APPLICATIONS

This application claims the benefit of Chinese Patent Application Serial No. 202510084179.1 filed on Jan. 17, 2025, the disclosure of which is incorporated herein by reference.

### TECHNICAL FIELD

The present disclosure pertains to medical devices. More particularly, the present disclosure pertains to medical devices for use in electrosurgical procedures.

### BACKGROUND

Electrosurgical procedures are widely used in modern medicine for cutting, coagulating, and ablating tissue. These procedures utilize electrical current to achieve precise tissue effects. Medical devices are used in endoscopic procedures for tissue removal and have been adapted for use with various energy modalities. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including electrosurgical devices, scissor ESD knife devices, and/or other medical devices and associated devices.

A first example may include a medical device including an elongate shaft, an end effector at a distal end of the elongate shaft, a fluid channel extending through the elongate shaft to the end effector, and a control shaft extending through the elongate shaft for controlling operation of the end effector.

Alternatively or additionally to any of the examples above, the end effector may be configured to rotate in response to rotation of the control shaft.

Alternatively or additionally to any of the examples above, the end effector may be configured to adjust between a closed position and an opened position in response to axial movement of the control shaft relative to the elongate shaft.

Alternatively or additionally to any of the examples above, the end effector may be configured to adjust between an opened position and a closed position and when the end effector is in the closed position, a fluid outlet of the fluid channel may be closed and when the end effector is in the opened position, the fluid outlet of the fluid channel may be open.

Alternatively or additionally to any of the examples above, the end effector may comprise a first jaw and a second jaw and when the end effector is in an opened position, the first jaw is spaced from the second jaw and when the end effector is in a closed position, the first jaw overlaps the second jaw.

Alternatively or additionally to any of the examples above, the end effector may define a fluid outlet path, and when the end effector is in an opened position, the fluid outlet path may be in fluid communication with the fluid channel and when the end effector is in a closed position, the fluid outlet path may be disconnected from fluid communication with the fluid channel.

Alternatively or additionally to any of the examples above, the control shaft may be electrically conductive and configured to provide electrical power to the end effector.

Alternatively or additionally to any of the examples above, the control shaft may be electrically insulated from the fluid channel.

Alternatively or additionally to any of the examples above, the medical device may further include a post coupled with the control shaft and electrically coupled with the end effector, wherein the post may define a first post channel of the fluid channel and a second post channel of the fluid channel.

Alternatively or additionally to any of the examples above, the end effector may define a first fluid outlet path and a second fluid outlet path, and when the end effector is in an opened position, the first fluid outlet path may be in fluid communication with the first post channel and the second fluid outlet path may be in fluid communication with the second post channel.

In an example, a medical device may include a handle, the handle comprising a fluid port, an electrical connector, and an actuator, an elongate shaft extending from the handle and an end effector at a distal end of the elongate shaft, the end effector is in fluid communication with the fluid port and is in electrical communication with the electrical connector, wherein actuation of the actuator may cause the end effector to adjust position.

Alternatively or additionally to any of the examples above, the actuation of the actuator to a first position may be configured to permit fluid from the fluid port to flow through the end effector and actuation of the actuator to a second position may be configured to prevent fluid flow through the end effector.

Alternatively or additionally to any of the examples above, the medical device may further include a fluid channel in fluid communication with the fluid port and extending through the elongate shaft to the end effector.

Alternatively or additionally to any of the examples above, the medical device may further include a control shaft extending through the elongate shaft, the control shaft may be coupled with the end effector and the electrical connector.

Alternatively or additionally to any of the examples above, the electrical connector may be electrically isolated from the fluid port.

In an example, a method may include delivering a cutting tool to a target site within a patient, where the cutting tool is located at distal end of an elongate shaft, delivering fluid to the target site through the elongate shaft of the cutting tool, applying electrical power to the cutting tool, and cutting tissue with the cutting tool using the electrical power.

Alternatively or additionally to any of the examples above, the fluid may be delivered to the cutting tool and the electrical power is applied to the cutting tool simultaneously.

Alternatively or additionally to any of the examples above, a control shaft may be coupled with an actuator and the cutting tool and the method may further comprise actuating the actuator from a first position in which the cutting tool is a first orientation configured to allow fluid to pass through the cutting tool to a second position in which the cutting tool is in a second orientation configured to prevent fluid from passing through the cutting tool.

Alternatively or additionally to any of the examples above, cutting tissue with the cutting tool may include spacing a first jaw of the cutting tool from a second jaw of the cutting tool, positioning the first jaw and the second jaw around tissue, and engaging tissue with the first jaw and the second jaw.

Alternatively or additionally to any of the examples above, the method may further include delivering fluid from the cutting tool while the first jaw is spaced from the second jaw.

The above summary of some embodiments is not intended to describe each disclosed embodiment or every implementation of the present disclosure. The Figures and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative medical system;

FIG. 2 is a schematic cross-section view of an illustrative medical device;

FIG. 3 is a schematic cross-section view of a portion of the medical device depicted in FIG. 2, taken from box-3;

FIG. 4 is a schematic perspective view of a distal portion of an illustrative medical device in a first configuration;

FIG. 5 is a schematic end view of the distal portion of the medical device depicted in FIG. 4 in the first configuration;

FIG. 6 is a schematic cross-section view of the distal portion of the medical device depicted in FIG. 4 in the first configuration;

FIG. 7 is a schematic perspective view of the distal portion of the medical device depicted in FIG. 4 in a second configuration;

FIG. 8 is a schematic cross-section of the distal portion of the medical device depicted in FIG. 4 in the second configuration; and

FIG. 9 is a schematic diagram of an illustrative method of using a medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

The present disclosure relates to medical devices for use in endoscopic submucosal dissection (ESD) procedures. Medical devices for use in ESD procedures include, but are not limited, to electrosurgical medical devices, electrosurgical graspers, biopsy tools, cutting tools, scissor ESD knives, and/or other suitable medical devices used for cutting, excising, dissecting, and/or cauterizing vessels and/or tissue. In one example, a scissor ESD knife may use high-frequency current to cut and/or cauterize cut vessels and/or tissue during an ESD procedure. In use, blood and carbonized tissue may pool or collect at or proximate jaws of the scissor ESD knife or a target site during a procedure and as such, water may be injected to the jaws and/or target site during the ESD procedure to clear the area of blood and carbonized tissue and/or for other purposes.

The concepts disclosed herein may include a medical device for use in ESD procedures and/or other suitable procedures that is configured to cut and/or cauterize tissue and/or vessels and inject fluid (e.g., liquid) to a target site. Although the concepts discussed herein may refer to a medical device configured as a scissor ESD knife, the concepts may be used with or may be incorporated into other suitable medical devices.

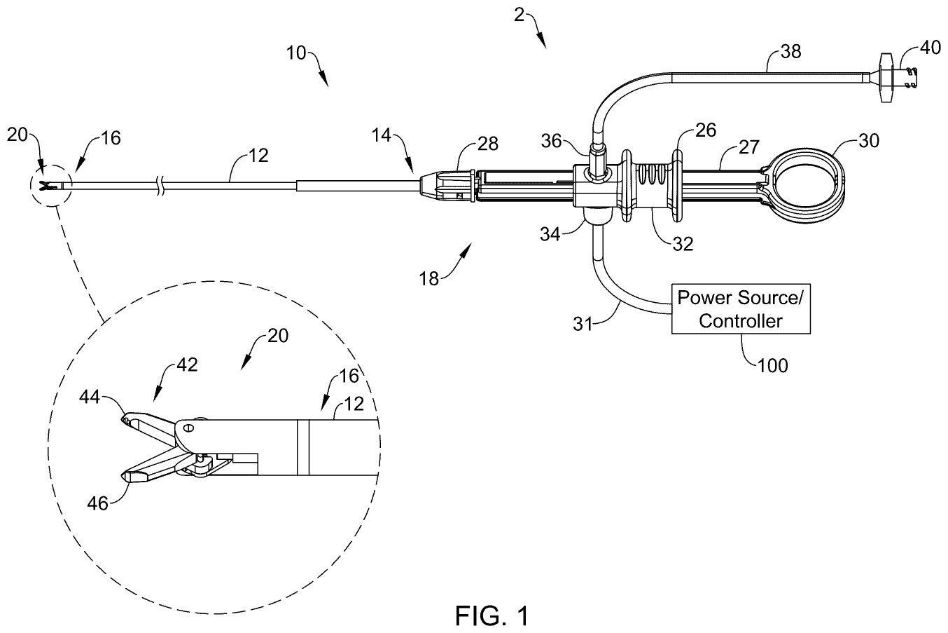

FIG. 1 depicts a schematic configuration of an illustrative medical system 2. The medical system 2 comprises a medical device 10 and a controller 100. In some examples, the medical device 10 may be a cutting tool, a grasper, a biopsy tool, a scissor ESD knife, and/or other suitable medical device. As depicted in FIG. 1, the medical device 10 may be an electrosurgical scissor ESD knife.

The medical device 10 may include a first shaft 12 having a proximal region 14 and a distal region 16 and an end effector 20 located at and/or distal of the distal region 16 of (e.g., a distal end of) the first shaft 12. A handle 18 of the medical device 10 may be coupled to the proximal region 14 of the first shaft 12, coupled with other suitable components of the medical device 10, and/or in communication with the end effector 20.

The end effector 20 may have any suitable configuration. In some examples, the end effector 20 may include one or more jaws. When the end effector 20 includes one or more jaws, the end effector 20 may include a first jaw and a second jaw configured to adjust between a first configuration (e.g., an opened configuration) and a second configuration (e.g., a closed configuration) in response to actuation of the handle 18. In some examples, the end effector 20 may be configured to rotate in response to actuation of the handle 18 (e.g., rotation of one or more portions of the handle 18). In some examples, the end effector 20 may be configured to rotate three-hundred-sixty degrees with the handle 18 or a portion thereof, but other suitable rotational limits, if any, are contemplated. In some examples, the end effector 20 may be electrically conductive or have one or more electrically conductive surfaces configured to apply current to vessels or tissue between the jaws.

The handle 18 may have any suitable configuration and may be formed from any suitable material discussed herein or otherwise. In some examples, the handle 18 may be coupled with a proximal end of the first shaft 12 or other suitable component of the medical device 10 and may operate as a graspable component or portion of medical device 10. For example, the handle 18 may include a first portion 26 (e.g., an actuator or control mechanism) which may be coupled with a control shaft (not depicted) coupled with the end effector 20 and a second portion 27 rotationally coupled with a third portion 28 of the handle 18. The third portion 28 of the handle 18 may be coupled with the proximal end or region 14 of the first shaft 12. In some examples, the first portion 26 and the second portion 27 may be translated rotationally relative to the third portion 28 of the handle 18 to rotationally adjust the end effector 20. In some examples, the first portion 26 of the handle 18 may be configured to translate longitudinally relative to the second portion 27 of the handle to adjust the end effector 20 between a first configuration and a second configuration. For example, the medical device 10 may be configured so that when the first portion 26 of the handle 18 is adjusted in a distal direction (e.g., a distal axial direction) relative to the second portion 27 and/or the third portion 28 of the handle 18, the end effector 20 may be in or adjusted to the first configuration configured to receive a vessel or tissue. When the first portion 26 is slid or positioned in a proximal direction (e.g., a proximal axial direction) relative to the second portion 27 and/or the third portion 28, the end effector 20 may be in or adjusted to the second configuration and configured to be engaging a vessel or tissue, or completely closed. As such, in some examples the first portion 26 may be configured to axially move to adjust the end effector 20 between the first configuration and the second configuration. Although the handle 18 is discussed with the first portion 26 being adjustable to adjust the end effector 20 between the first configuration and the second configuration, it is contemplated that the second portion 27 and/or other suitable portion of the handle 18 may be adjustable to adjust the end effector 20 between the first configuration and the second configuration.

The handle 18 may include a number of structural features. For example, the first portion 26 and/or the second portion 27 of the handle 18 may include one or more finger holes 30 and/or finger holds 32 to help assist in grasping and/or positioning the handle 18. In some examples, the finger holes 30 may be rings configured to receive a user's finger. In some examples, the finger holds 32 may be or may include one or more surfaces configured to receive one or more fingers of the user and facilitate laterally adjusting the finger hold 32 or a component coupled thereto. In one example, the finger holds 32 may have proximal and distal lips that may engage one or more fingers of the user grasping the finger hold 32 when longitudinally adjusting the finger hold 32. In one example, the first portion 26 of the handle 18 may include a finger hold 32 configured to receive two fingers of a user and the second portion 27 of the handle 18 may include the finger hole 30 for receiving a finger (e.g., a thumb or other suitable finger) of a user to facilitate adjusting or otherwise moving the first portion 26 with (e.g., moving rotationally with) and/or relative to (e.g., moving longitudinally relative to) the second portion 27 of the handle 18. Alternatively, the first portion 26 of the handle 18 may include a single finger hole 30 and the second portion 27 of the handle 18 may include the finger hold 32. Other suitable features and/or configurations of the handle 18 are contemplated.

The first portion 26 of the handle 18 may include one or more ports. In some examples, the handle 18 may include a first port 34, a second port 36, and/or one or more other suitable ports. Although the ports are depicted in FIG. 1 as being part of the first portion 26, one or more ports may be located at other suitable portions of the handle 18. The first port 34 may be used, for example, as an access point to apply and/or deliver electrosurgical current to a control shaft and/or the end effector 20 and, optionally, receive a return current. The second port 36, may be used, for example, as a fluid port configured to couple with a fluid source to receive and/or deliver fluid to the end effector 20 and/or a target site during a procedure. The second port 36 may be directly coupled with a fluid source or coupled with a fluid source via one or more tube 38 and/or connectors 40. Other suitable configurations of the first port 34, the second port 36, and/or other suitable ports of medical device 10 are contemplated.

The connector 40 configured to couple with a fluid source may be any suitable type of connector. For example, the connector 40 may be configured as male connector, a female connector, a luer connector, a threaded connector, a bayonet connector, and/or other suitable type of connector. In one example and as depicted in FIG. 1, the connector 40 may be configured as a female luer connector component.

The medical device 10 and/or the medical system 2 may include an outer shaft or sheath through which the first shaft 12 and/or the end effector 20 may extend. In some examples, the one or more outer shafts or sheaths or other suitable elongate tubes may be guide sheaths, insulating sheaths, and/or other suitable types of shafts or sheaths.

The first shaft 12 may have any suitable configuration. In some examples, the first shaft 12 may be an electrically conductive shaft and/or an electrically insulative shaft, but other suitable configurations are contemplated. For example, the first shaft 12 may have one or more layers of electrically conductive material, one layers of electrically insulative materials, or one or more layers of electrically conductive materials and one or more layers of electrically insulative materials. In one example, an outer layer defining an outer surface of the first shaft 12 may be a layer of insulative material and an inner layer of the first shaft 12 may be an electrically conductive material. In one example, the first shaft 12 may be formed only of electrically insulating material.

In some example configurations, the first shaft 12 may be tubular and configured to bend along the length of the first shaft 12 as the first shaft 12 passes through one or more body cavities or lumens. In one example, when the first shaft 12 is formed from or as a tube, the first shaft 12 may define a lumen configured to receive a control shaft coupled with the end effector 20.

The end effector 20 may have any suitable configuration configured to grasp and/or cut a vessel or tissue and apply electrical current to the vessel or tissue. In some examples, the end effector 20 may include a jaw assembly 42 having a first jaw 44 and a second jaw 46. In some examples, the first jaw 44 and the second jaw 46 may adjust between a first configuration (e.g., an opened configuration, as depicted for example in FIG. 1) and a second configuration (e.g., a closed configuration) in response to adjustment of the first portion 26 of the handle 18 relative to the second portion 27 and/or the third portion 28 of the handle 18. As discussed in greater detail below, the jaw assembly 42 may be configured to receive electrical power (e.g., electrical current) via a control shaft and/or one or more wires in electrical communication with the controller 100.

The first port 34 may be configured to electrically couple an electrical connector 31 with the end effector 20, where the end effector 20 may be configured to provide electrosurgical current to a vessel or tissue of the subject. The electrical connector 31 may be coupled with one or more power sources or controllers 100 configured to supply electrosurgical current to the medical device 10 (e.g., in monopolar and/or bipolar configurations). The power source or controller 100 may be configured to facilitate operation of the medical device 10.

The power source or controller 100 may be and/or may include any suitable computing device configured to process data of or for the medical device 10. In some cases, the power source or controller 100 may be configured to apply power (e.g., current) to the medical device 10 in response to one or more user inputs, control signals, conditions of the medical device, conditions of the subject, a schedule, and/or one or more other prompts. Further, one or more components of the medical device 10 may incorporate one or more computing devices similar to or having components similar to the power source or controller 100.

The illustrative power source or controller 100 may include, among other suitable components, one or more processors, memory, and/or one or more I/O units. Example other suitable components of the power source or controller may include, but are not limited to, communication components, a touch screen, selectable buttons, a housing, an electrical connection to line power, and/or other suitable components of a power source or controller. One or more components of the power source or controller 100 may be separate from the components of the medical device 10 and/or incorporated into the components of the medical device 10.

The processor of the power source or controller 100 may include a single processor or more than one processor working individually or with one another. The processor may be configured to receive and execute instructions, including instructions that may be loaded into memory. Example components of the processor may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory of the power source or controller 100 may include a single memory component or more than one memory component each working individually or with one another. Example types of memory may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory may be or may include a non-transitory computer readable medium. The memory may include instructions stored in transitory and/or non-transitory state on a computer readable medium that may be executable by the processor to cause the processor to perform one or more of the methods and/or techniques, or a portion thereof, described herein.

The I/O units of the power source or controller 100 may include a single I/O component or more than one I/O component each working individually or with one another. Example I/O units may be or may include any suitable types of communication hardware and/or software including, but not limited to, communication ports configured to communicate with electronic or electrical components of the medical device 10 and/or with other suitable devices or systems. Example types of I/O units may include, but are not limited to, electrical outlets, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units.

The medical system 2 may include a user interface configured to communicate with the power source or controller 100 via one or more wired or wireless connections. The user interface may include one or more display devices, one or more input devices, one or more output devices, and/or one or more other suitable features.

The display device, when included with the user interface, may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s), when included with the user interface, may be and/or may include any suitable components and/or features for receiving user input via the user interface. Example input device(s) include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s), when included with the user interface may be and/or may include any suitable components and/or features for providing information and/or data to users and/or other computing components. Example output device(s) include, but are not limited to, displays, speakers, vibration systems, tactile feedback systems, optical outputs, cables, lights, and/or other suitable output devices.

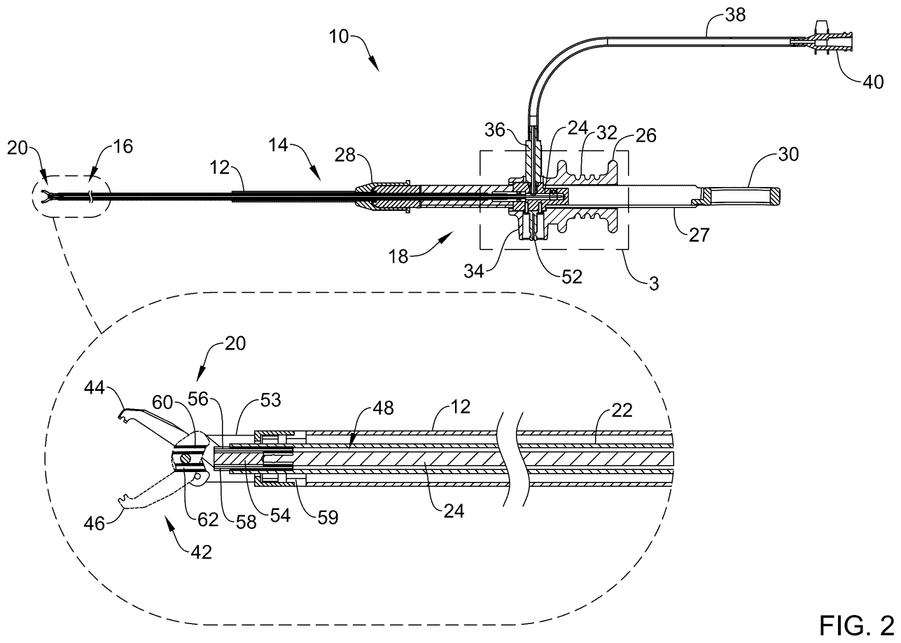

FIG. 2 depicts a schematic cross-section view of the medical device 10 depicted in FIG. 1, where the cross-section has been taken along a longitudinal axis of the medical device. At the jaw assembly 42 of the end effector 20, the second jaw 46 is shown in broken lines as it would not typically be depicted in the cross-section view of FIG. 2.

As discussed, the handle 18 may include the first port 34 and the second port 36. The first port 34 may be configured to couple with or facilitate coupling the medical device 10 with the controller 100. In some examples, the handle 18 may include an electrical connector 52. The electrical connector 52 may be any suitable connector configured to electrically communicate or otherwise electrically couple with the controller 100 (e.g., via the electrical connector 31 and/or in one or more other suitable manners). In some examples, the electrical connector 52 may be a plug, socket, and/or other suitable component.

As depicted in the magnified portion of FIG. 2, the medical device 10 may include the first shaft 12, a second shaft 22, and a control shaft 24. In some examples, the first shaft 12, the second shaft 22, and the control shaft 24 may be individually and/or collectively referred to as “an elongate shaft”). In some examples, one or more of the first shaft 12, the second shaft 22, and the control shaft 24 may extend from the handle 18 to the end effector 20. The first shaft 12 may be coupled with the end effector 20 using a coupling cuff 59 and/or other suitable component(s).

The second shaft 22 may have any suitable configuration. In some example configurations, the second shaft 22 may be tubular, may be elongated, may have a solid cross-section, and/or may have one or more other suitable configurations. In some examples, the second shaft 22 may be configured to bend along the length of the second shaft 22 as the second shaft 22 passes through one or more body cavities or lumens. In one example, when the second shaft 22 may be formed from or as a tube, the second shaft 22 may define a lumen configured to receive the control shaft 24 coupled with the end effector 20. In some examples, an inner surface of the second shaft 22 defining the lumen thereof may at least partially define one or more fluid lumens 48 (e.g., a fluid channel) extending through the first shaft 12 and/or the second shaft 22, between the second shaft 22 and the control shaft 24.

The second shaft 22 may extend from the handle 18 to the end effector 20 and couple to any suitable components of the medical device 10. In some examples, a proximal end of the second shaft 22 may be configured to couple with the second portion 27 of the handle 18 so as to rotate with the first portion 26 and the second portion 27, but other suitable configurations are contemplated. A distal end of the second shaft 22 may couple with the end effector 20. In some examples, the distal end of the second shaft 22 may be coupled with a clevis 53 of the end effector 20. Other suitable couplings of the second shaft 22 with other components of the medical device 10 are contemplated.

The control shaft 24 (e.g., a control wire) may have any suitable configuration. In some example configurations, the control shaft 24 may be tubular, may be elongated, may have a solid cross-section, and/or may have one or more other suitable configurations. In some examples, the control shaft 24 may be configured to bend along the length of the control shaft 24 as the control shaft 24 passes through one or more body cavities or lumens. In one example, the control shaft 24 may be formed from or as a solid wire. In some examples, an outer surface of the control shaft 24 may at least partially define the fluid lumen 48. In some examples, the control shaft 24 may include one or more coatings or layers of insulative material thereon that define the fluid lumen 48 to isolate the fluid lumen 48 and fluid therein from electrical power that may pass through the control shaft 24.

The control shaft 24 may extend from the handle 18 to the end effector 20 and couple to any suitable components of the medical device 10. In some examples, a proximal end of the control shaft 24 may be configured to couple with or be in electrical communication with the electrical connector 52, along with being coupled with the first portion 26 of the handle 18 so as to rotate with and/or longitudinally translate with the first portion 26. A distal end of the control shaft 24 may couple with the end effector 20 such that electrical current may travel from the electrical connector 52, through the control shaft 24, and to the end effector 20. In some examples, the distal end of the control shaft 24 may be connected to a post 54 of the end effector 20 that is operatively coupled with the jaw assembly 42 to adjust the jaw assembly 42 between the first configuration and the second configuration as the first portion 26 of the handle 18 translates axially and/or longitudinally relative to the second portion 27 of the handle 18 and in response the control shaft 24. In some examples, the control shaft 24 may be coupled to the post 54 with a threaded connection, a weld, and/or other suitable type of connection. Other suitable couplings of the control shaft 24 with other components of the medical device 10 are contemplated.

The fluid lumen 48 may have any suitable configuration. In some examples, the fluid lumen 48 may extend from the second port 36 through the second shaft 22. In some examples, the fluid lumen 48 may extend circumferentially around the control shaft 24 and have an outer boundary defined by an inner surface of the second shaft 22, but other suitable configurations are contemplated, including, but not limited to, one or more fluid lumens 48 each in fluid communication with the second port 36 and extending along the second shaft 22 and extending less than circumferentially around the control shaft 34.

The post 54 of the end effector 20 may have one or more post channels. In some examples, the post 54 may have a first post channel 56 and a second post channel 58 extending longitudinally through the post 54. At a proximal end of the post 54, the first post channel 56 and the second post channel 58 may be in fluid communication with the fluid lumen(s) 48 extending along the second shaft 22 and the control shaft 24. At a distal end of the post 54, the first post channel 56 and the second post channel 58 of the post 54 may be aligned within one or more channels of the jaw assembly 42. In one example, when the jaw assembly 42 is in an opened configuration, as depicted for example in FIG. 2, the first post channel 56 may be aligned with a first outlet channel 60 (e.g., a first fluid outlet channel or first fluid outlet path) of the jaw assembly 42 and the second post channel 58 may be aligned with a second outlet channel 62 (e.g., a second fluid outlet channel or second fluid outlet path) of the jaw assembly 42, where the first outlet channel 60 and/or the second outlet channel 62 may define fluid outlet paths through the jaw assembly 42. Other suitable configurations of the lumens or channels of the post 54 and/or the jaw assembly 42 are contemplated.

The first shaft 12, the handle 18, the end effector 20, the second shaft 22, the control shaft 24, the electrical connector 52, and/or other components of the medical device 10 may be formed from any suitable materials. Example suitable materials include, but are not limited to, polymers, metals, metal alloys, metal-polymer composites, and/or other suitable materials. Some example suitable polymers include, but are not limited to, polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, polyethylene (PE), Marlex high-density polyethylene, Marlex low-density polyethylene, linear low density polyethylene (for example, REXELL®), polyethylene terephthalate (PET), polyetheretherketone (PEEK), polyimide (PI), polyetherimide (PEI), polyphenylene sulfide (PPS), polyphenylene oxide (PPO), polysulfone, nylon, perfluoro(propyl vinyl ether) (PFA), other suitable materials, or mixtures, combinations, copolymers thereof, polymer/metal composites, and the like. Some example suitable metals and/or metal alloys include, but are not limited to, stainless steel, such as 304V, 304L, and 316L stainless steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like.

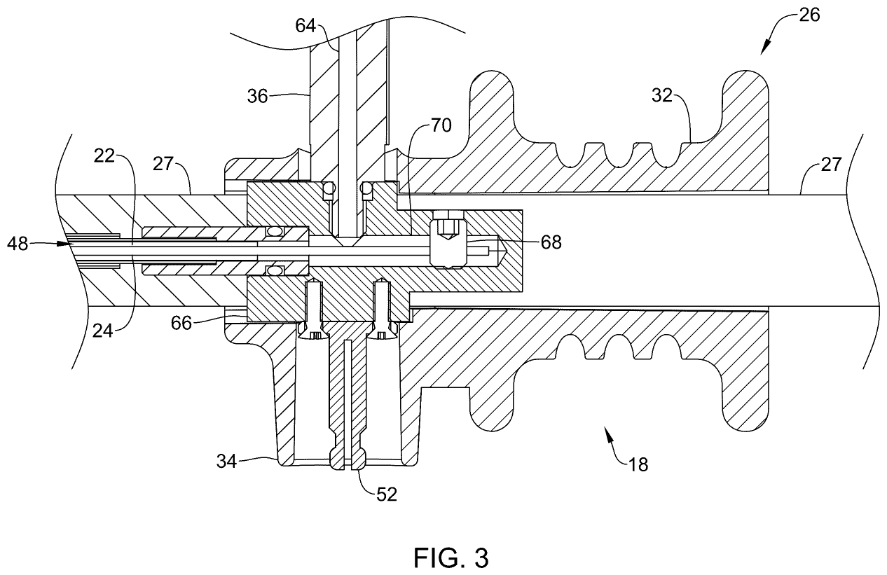

FIG. 3 depicts a schematic cross-section view of a portion of the handle 18, taken from box-3 in FIG. 2. The first portion 26 of the handle 18 is depicted in FIG. 3 as being configured to adjust longitudinally relative to the second portion 27 of the handle 18.

The first portion 26 of the handle 18 may include the finger hold 32, the first port 34, the second port 36, the electrical connector 52, and a manifold or carrier 66, among other suitable components. In some examples, the electrical connector 52 may be electrically and/or mechanically coupled with the carrier 66. Further, as depicted in FIG. 3, the second port 36 may be separable from one or more components of the first portion 26 of the handle 18, but it is contemplated that the second port 36 may be monolithically formed with at least one other component of the first portion 26 of the handle 18.

The carrier 66 may have any suitable configuration for coupling with and/or receiving the control shaft 24, receiving fluid from the second port 36 through a fluid channel 64, and electrically connecting with the electrical connector 52 to pass electrical power from the electrical connector 52 to the control shaft 24. The carrier 66 may include or receive a coupler 68 configured to engage the carrier 66 and the control shaft 24. In some examples, the coupler 68 may be electrically conductive and engage an electrically conductive portion of the control shaft 24 with an electrically conductive surface of the coupler 68 to transfer electrical current from the electrical connector 52 to the control shaft 24. Further, the carrier 66 may define a reservoir 70 configured to receive the fluid from the fluid channel 64 of the second port 36. In some examples, the reservoir 70 may be part of the fluid lumen 48. Alternative or additional configurations of the carrier 66 are contemplated.

The reservoir 70 of the carrier 66 may be electrically isolated from the electrically conductive components or electrically conductive portions of components of the first portion 26 of the handle 18. In some examples, a portion of the carrier 66 defining the reservoir 70 may be coated with or have a layer of electrically insulative material so as to isolate fluid passing through the second port 36 and into the reservoir 70 from electrical power passing through the carrier 66 and/or components or other portions of the first portion 26 of the handle 18.

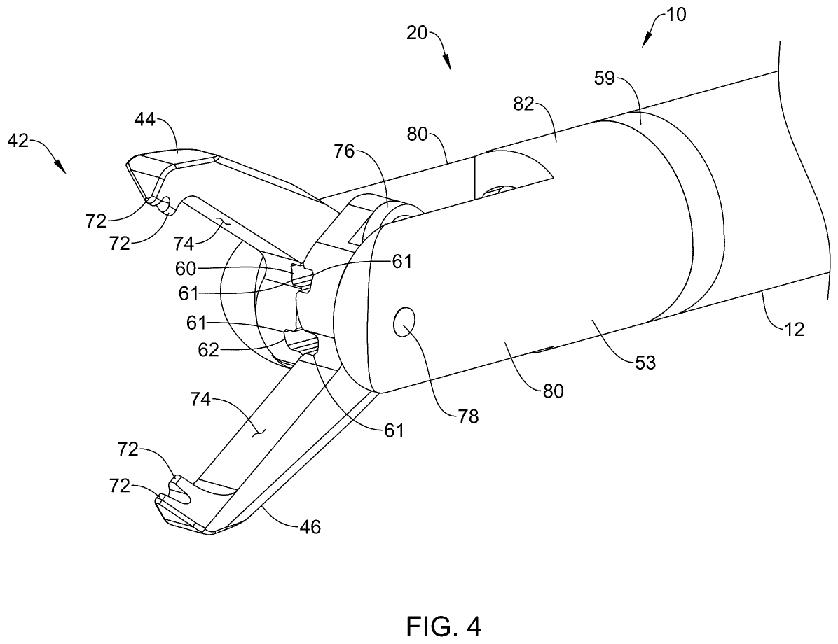

FIGS. 4-8 depict views of a distal end of the medical device 10. As discussed, the jaw assembly 42 may include the first outlet channel 60 and the second outlet channel 62. In some examples, the first jaw 44 and the second jaw 46 of the jaw assembly 42 may form or define a portion (e.g., a half or other suitable portion) of one or both of the first outlet channel 60 and the second outlet channel 62. Other suitable configurations of the first outlet channel 60 and/or the second outlet channel 62 are contemplated including, but not limited to, one or more of the first outlet channel 60, the second outlet channel 62 and/or other suitable outlet channels being entirely within (e.g., entirely defined by) one of the first jaw 44 and the second jaw 46 and/or formed with a portion of the jaw assembly 42 and portion of one or more other component of the end effector 20.

FIG. 4 is a schematic perspective view of the distal end of the medical device 10, depicting the end effector 20 in the first configuration. When the jaw assembly 42 is in the first configuration, the first jaw 44 and the second jaw 46 may be separated from one another and form and/or define the first outlet channel 60 and the second outlet channel 62, as depicted for example in FIG. 4, such that the fluid outlets are open and fluid may be outputted through the jaw assembly 42. In some examples and as discussed, the first jaw 44 and the second jaw 46 may each form or define a portion (e.g., a half) of one or both of the first outlet channel 60 and the second outlet channel 62. In one example, each of the first jaw 44 and the second jaw 46 may include one or more indentations 61 configured to align with one of the indentations 61 on the other one of the first jaw 44 and the second jaw 46 to define an outlet channel when the jaw assembly is in the first configuration. Other suitable configurations of the first outlet channel 60, the second outlet channel 62, and/or other outlet channels of the jaw assembly 42 are contemplated.

Among other suitable components, the end effector 20 may include the jaw assembly 42 with the first jaw 44 and the second jaw 46, one or more one or more linkages 76 pivotally coupled with the jaw assembly, the clevis 53 coupled with the jaw assembly using a pin 78, and the post 54 coupled with the one or more linkages 76 such that the linkages may pivot relative to the post 54. In some examples, the coupling cuff 59 coupling the first shaft 12 with the clevis 53 may be part of the end effector 20, but it is contemplated that the coupling cuff 50 may be separate from the end effector 20. Although the clevis 53 may have other suitable configurations, the clevis 53 may include a pair of arms 80 extending in a distal direction from a proximal portion 82 (e.g., a cylindrical proximal portion). Although other suitable examples are contemplated, example suitable couplings between a shaft, clevis, linkages, and jaws are described in U.S. Pat. No. 7,762,960 titled BIOPSY FORCEPS ASSEMBLIES filed on May 13, 2005, which is hereby incorporated by reference in its entirety for any and all purposes.

The first jaw 44 and the second jaw 46 may have any suitable configuration. In some examples, the first jaw 44 and the second jaw 46 may have the same or similar features, but other suitable configurations are contemplated. Some example features of the first jaw 44 and the second jaw 46 include, but are not limited to, one or more teeth 72 configured to engage tissue, one or more tissue engaging surfaces 74, and/or other suitable features. In some examples, the teeth 72, the tissue engaging surfaces 74, and/or other portions of the jaw assembly 42 may be electrically conductive and electrically coupled with the control shaft 24 to apply electrical power to vessels and/or tissue.

In the configuration of the medical device 10 depicted in the Figures, when the first portion 26 of the handle 18 is advanced in a distal direction relative to the second portion 27 of the handle 18, the post 54 advances in the distal direct causing the linkages 76 to apply a longitudinal force to a proximal end of the jaw assembly 42 and resulting in the first jaw 44 and the second jaw 46 pivoting about the pin 78 and separating from one another to move into the first configuration, as depicted in FIG. 4. However, it is contemplated, that end effector 20, the control shaft 24, and/or the handle 18 may be configured such that movement of the first portion 26 of the handle in the proximal direction (e.g., a proximal axial movement) or other suitable movement of one or more components of the handle 18 cause the jaw assembly 42 to adjust to the first configuration. When in the first configuration, fluid (e.g., water and/or other suitable fluid) may be outputted through the first outlet channel 60 and/or the second outlet channel 62 to clear debris from or proximate the jaw assembly 42.

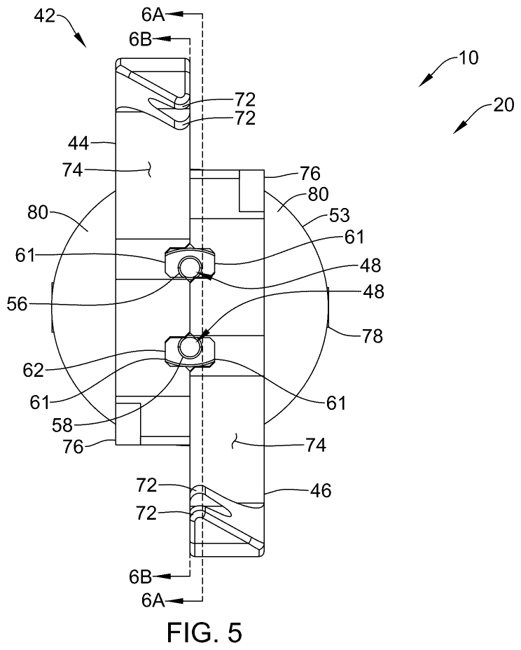

FIG. 5 depicts a schematic end view of the portion of the medical device 10 depicted in FIG. 4, with the end effector 20 in the first configuration. As depicted in FIG. 5, the first outlet channel 60 may align with the first post channel 56 and the fluid lumen 48 when the jaw assembly 42 is in the first configuration to allow fluid to exit the medical device 10 through the outlets of the jaw assembly 42. Similarly, the second outlet channel 62 may align with the second post channel 58 and the fluid lumen 48 when the jaw assembly 42 is in the first configuration. As such, fluid may be delivered to the target site and/or the first jaw 44 and/or the second jaw 46 to clear debris when the jaw assembly is in the first configuration.

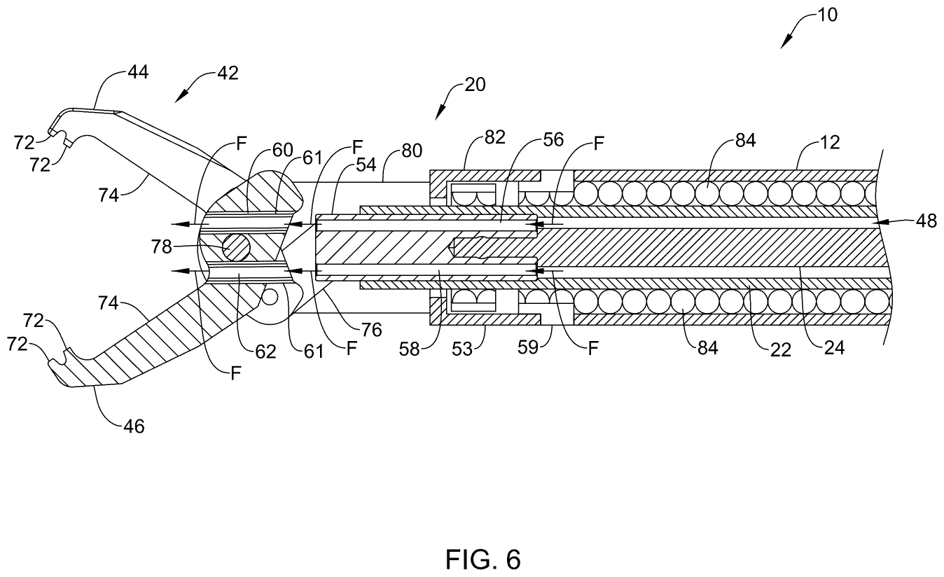

FIG. 6 depicts a schematic cross-section view of the portion of the medical device 10 depicted in FIG. 4, with the end effector 20 in the first configuration. The cross-section view depicted in FIG. 6 is taken along line 6A-6A for a majority of a length of the depicted portion of the medical device 10 and taken along line 6B-6B for a length of the depicted portion of the medical device 10 at the second jaw 46.

As depicted in FIG. 6, the first shaft 12 may extend over the second shaft 22. In some examples, a spring tube 84 may be positioned between the first shaft 12 and the second shaft 22 to provide stability to the distal end or portion of the medical device 10. The second shaft 22 may extend over the control shaft 24 and define the fluid lumen 48 therebetween. Other suitable configurations of the fluid lumen 48 are contemplated.

As discussed, the fluid lumen 48 may be aligned with the first post channel 56 and the second post channel 58. When the jaw assembly 42 is in the first configuration, the first outlet channel 60 and the second outlet channel 62 of the jaw assembly 42 may be formed and aligned with the first post channel 56 and the second post channel 58, respectively. In some examples, the fluid lumen 48, the first post channel 56, the second post channel 58, the first outlet channel 60, and the second outlet channel 62 may form one or more fluid channels that may be opened or closed depending on the configuration of the jaw assembly 42. As such, when the jaw assembly 42 is in the first configuration, the fluid channel may be opened and fluid may flow from the fluid lumen 48, through the first post channel 56 and the second post channel 58, and through the first outlet channel 60 and the second outlet channel 62, as represented by arrows F in FIG. 6.

A gap may exist between the post 54 and the jaw assembly 42 such that a distal end of the first post channel 56 and a proximal end of the first outlet channel 60 and/or, similarly, between a distal end of the second post channel 58 and a proximal end of the second outlet channel 62. When the gap between the post 54 and the jaw assembly 42 exists, fluid therethrough may travel at a speed such that the fluid jumps the gap and exits the medical device 10 at a distal end of the first outlet channel 60 and/or the second outlet channel 62. In some examples, there may be no gap between the post 54 and the jaw assembly 42 or one or more components (e.g., tubes, deflectors, shafts, etc.) may bridge the channels of the post 54 with the channels of the jaw assembly 42.

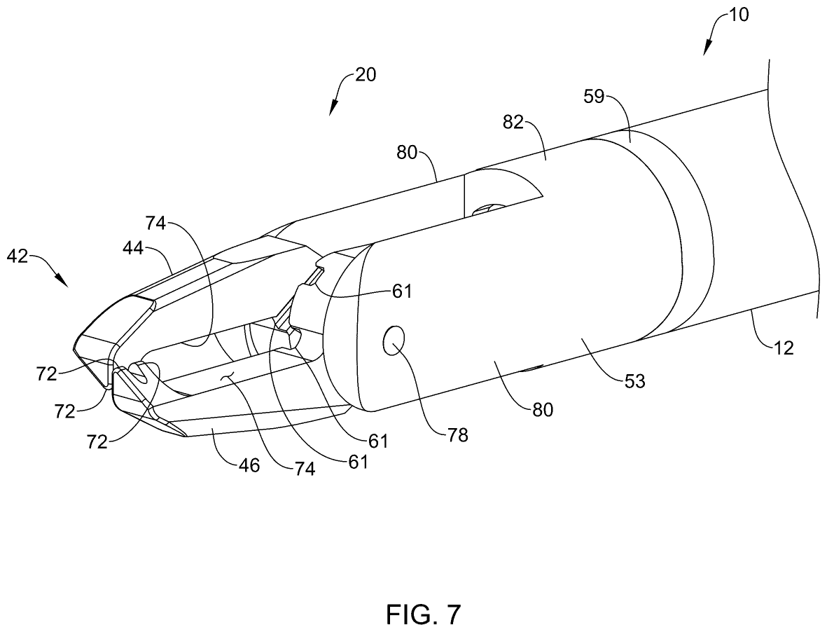

FIG. 7 is a schematic perspective view of the distal end of the medical device 10 depicted in FIG. 4 with the end effector 20 in the second configuration (e.g., a closed configuration). In some examples, the first portion 26 of the handle 18 may be advanced in a longitudinal proximal direction (not shown in FIG. 7), which moves the control shaft 12 and the post 54 proximally in a manner that pulls the linkages 76 in the proximal direction and causes the first jaw 44 and the second jaw 46 to pivot about the pin 78 and adjust to the second configuration. When the jaw assembly 42 is in the second configuration, the first jaw 44 and the second jaw 46 may overlap, engage one another, and/or include portions that are proximate one another. In one example, the teeth 72 of the first jaw 44 may overlap the teeth 72 of the second jaw 46 and the tissue engaging surfaces 74 of the jaw assembly 42 may be positioned proximate one another when the jaw assembly 42 is in the second configuration, as depicted for example in FIG. 7. In some examples, the tissue engaging surfaces 74 of the first jaw 44 an the second jaw 46 may contact one another in the second configuration when there is no tissue therebetween. In some examples, when jaw assembly 42 is in the second configuration, an electrical current (e.g., an electrosurgical current) may be applied to vessels or tissue between the first jaw 44 and the second jaw 46 via the teeth 72, the tissue engaging surfaces 74, and/or other suitable portions of the jaw assembly 42. Other suitable configurations of the first jaw 44 and the second jaw 46 in the second configuration are contemplated.

When the jaw assembly 42 is in the second configuration, the first outlet channel 60 and the second outlet channel 62 may be pivoted or rotated with the first jaw 44 and the second jaw 46 such that an outlet of the fluid channel is disconnected from the fluid channel extending proximal thereof or otherwise closed to block or prevent fluid from exiting the medical device 10 through the jaw assembly 42. Additionally or alternatively, the components defining the first outlet channel 60 and the second outlet channel 62 of the first jaw 44 and the second jaw 46 may be misaligned with components defining the first outlet channel 60 and the second outlet channel on the other of the first jaw 44 and the second jaw 46. In one example and as depicted in FIG. 7, when the jaw assembly 42 is in the second configuration, indentations 61 of the first jaw 44 may be rotated with the first jaw 44 and the indentations 61 of the second jaw 46 may be rotated with the second jaw 46 such that the indentations 61 of the respective jaws are misaligned and do not form the first outlet channel 60 and the second outlet channel 62.

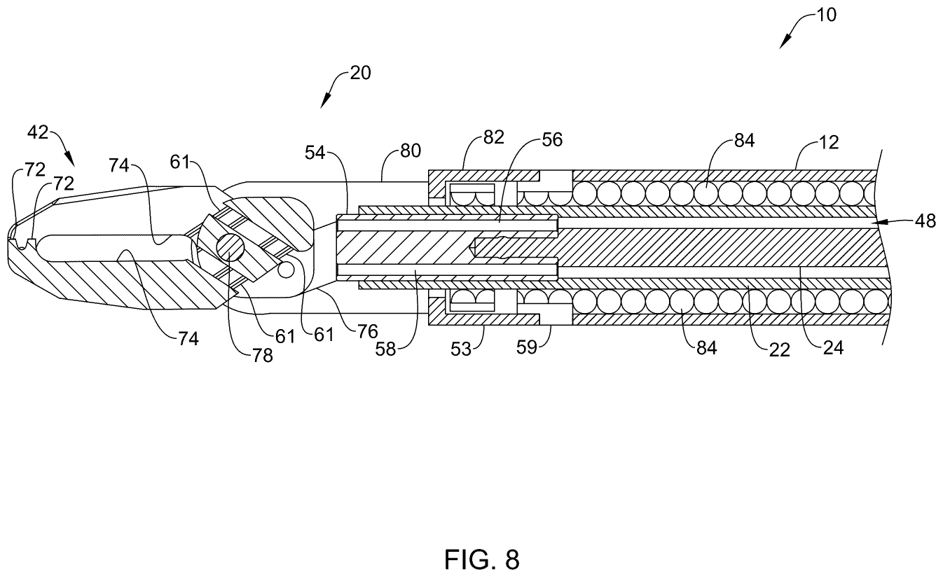

FIG. 8 depicts a schematic cross-section view of the distal portion of the medical device 10 depicted in FIG. 6, with the end effector 20 in the second configuration. The cross-section view depicted in FIG. 8 may be similar to the cross-section view of the medical device 10 that is depicted in FIG. 6.

As discussed, the fluid lumen 48 may be aligned with the first post channel 56 and the second post channel 58. When the jaw assembly 42 is in the second configuration, however, the first outlet channel 60 and the second outlet channel 62 of the jaw assembly 42 may be misaligned with first post channel 56 and the second post channel 58 such that fluid from the fluid lumen 48 is prevented from passing through the first outlet channel 60 and/or the second outlet channel 62. In some examples, portions of the first jaw 44 and/or the second jaw 46 (e.g., as depicted in FIG. 8), portions of the linkages 76, or other portions of the end effector 20 may block a flow path through the first post channel 56 and/or the second post channel 58 such that no fluid exits the first post channel 56 and/or the second post channel 58 when the jaw assembly 42 is in the second configuration. Further, as depicted in FIG. 8, when the first outlet channel 60 and the second outlet channel 62 are formed from indentations 61 in each of the first jaw 44 and the second jaw 46, the indentations 61 of one jaw may be misaligned with indentations 61 on the other jaw and misaligned with the first post channel 56 and the second post channel 58 such that the first outlet channel 60 and the second outlet channel 62 may not be formed and the indentations 61 are misaligned with the first post channel 56 and the second post channel 58. The arrows F provide an illustrative representation of the flow of fluid through the distal portion of the medical device 10 when the end effector 20 (e.g., the jaw assembly 42) is in the second configuration.

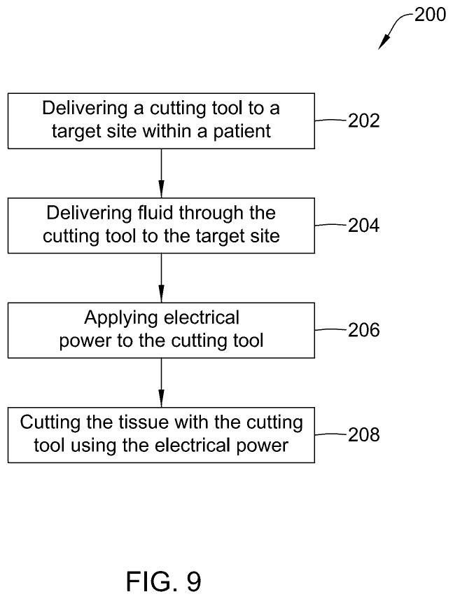

FIG. 9 depicts a schematic diagram of an illustrative method 200 of using a medical device. The method 200 may be configured to cut, excise, and/or cauterize tissue at a target site using the medical device, while clearing debris and/or blood from the target site before, during, and/or after the cutting, excising, and/or cauterizing of the tissue. The medical device used in the method may be or may include a cutting tool (e.g., a biopsy tool, a grasper, a scissors ESD knife as discussed herein, and/or other suitable cutting device). In some examples, the cutting tool may include an end effector comprising a jaw assembly at a distal end of one or more elongate shafts coupled with a handle.

The method 200 may include delivering 202 the cutting tool to a target site (e.g., a target location) within a subject (e.g., a patient). In some examples, the cutting tool may be delivered to the target site over a guidewire. In some examples, an endoscope may be inserted to the target site and the cutting tool may be delivered to the target site by inserting the cutting tool into the endoscope and passing the cutting tool through the endoscope to the target location. In some examples, a distal end of the cutting tool, which may include the jaw assembly having one or more jaws movable between a first configuration (e.g., an opened configuration) and a second configuration (e.g., a closed configuration), may be delivered to and/or otherwise positioned at the target site. The jaw assembly may be in the second configuration during delivery thereof to the target site, but other suitable configurations are contemplated.

Once the cutting tool is positioned at the target site, fluid (e.g., liquid, saline, water, contrast, etc.) may be delivered 204 to the target site through the elongate shaft. In some examples, the jaw assembly of the cutting tool may be adjusted to a first configuration (e.g., by adjusting an actuator or a control mechanism at the handle of the cutting tool to a first position) to space a first jaw of the jaw assembly from a second jaw of the jaw assembly and to align one or more outlet channels defined by the jaws of the jaw assembly with a fluid lumen along the elongate shaft of the medical device. Once the outlet channels of the jaw assembly are aligned with the fluid lumen, fluid may be delivered to the target site via the fluid lumen and the outlet channels of the jaw assembly. In some examples, fluid may be provided to the outlet channels via a fluid source coupled with a fluid port at or proximate a handle of the cutting tool, but other suitable configurations for providing fluid are contemplated. In some examples, the fluid from the fluid lumen may be applied to the target site without passing through the jaw assembly.

With the jaw assembly in the first configuration, the spaced apart jaws of the jaw assembly may be positioned around one or more vessels or tissue to be cut, excised, and/or cauterized. With the vessel(s) or tissue positioned within and engaging the jaw assembly, electrical power (e.g., current, voltage, etc.) may be applied 206 to the cutting tool (e.g., to the jaw assembly). The electrical power may be applied to the cutting tool simultaneously with the application of fluid to the target site or before and/or after applying fluid to the target site. In some examples, the electrical power may be applied to the jaw assembly through applying electrical power to an electrical connector at the handle of the cutting tool which may be electrically coupled with a control shaft electrically and mechanically coupled with the jaw assembly. In some examples, the jaws of the jaw assembly may include teeth and/or a tissue engaging surface, which may be electrically conductive and configured to apply the electrical power to the tissue at, proximate, and/or within the jaw assembly. In some examples, portions of the jaw assembly configured to engage tissue may be electrically conductive, while portions of the jaw assembly not configured to engage tissue (e.g., the outlet channels, etc.) may be electrically insulative, but other suitable configurations of the jaw assembly are contemplated.

Before during or after applying 206 the electrical power to the cutting tool, the vessel(s) and/or tissue within or at the jaw assembly may be cut 208 using the electrical power provided to the cutting tool. In some examples, the jaw assembly may be adjusted to the second configuration (e.g., by adjusting the actuator or control mechanism at a handle portion of the cutting tool to a second position) to close the jaws of the jaw assembly pinch or cut the vessel(s) and/or tissue therebetween with the jaws and/or the electrical power provided to the jaw assembly. In some examples, the electrical power applied to the vessel(s) and/or the tissue by the jaws of the jaw assembly may cut and cauterize the vessel(s) and/or the tissue between the jaws. Other suitable configurations for cutting the vessel(s) and/or the tissue are contemplated.

After the tissue has been cut, excised, and/or cauterized, the jaw assembly may be adjusted to the first configuration and the fluid may be outputted from outlet channels in the jaw assembly or along one or more other suitable paths to clear debris and/or blood from the target site and/or the jaws of the jaw assembly. The steps discussed herein and/or other steps for cutting, excising, and/or cauterizing tissue may be repeated until the desired vessel(s) and/or tissue have been cut, excised, and/or cauterized.

The schematic methods and techniques discussed herein may be used together, unless expressly indicated otherwise. Further, the order of implementation of the steps discussed herein may be in other suitable orders, unless expressly indicated otherwise, may include one or more intervening steps discussed herein or otherwise.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical device comprising:
an elongate shaft;
an end effector at a distal end of the elongate shaft;
a fluid channel extending through the elongate shaft to the end effector; and
a control shaft extending through the elongate shaft for controlling operation of the end effector.

2. The medical device of claim 1, wherein the end effector is configured to rotate in response to rotation of the control shaft.

3. The medical device of claim 1, wherein the end effector is configured to adjust between a closed position and an opened position in response to axial movement of the control shaft relative to the elongate shaft.

4. The medical device of claim 1, wherein:
the end effector is configured to adjust between an opened position and a closed position, and
when the end effector is in the closed position, a fluid outlet of the fluid channel is closed and when the end effector is in the opened position, the fluid outlet of the fluid channel is open.

5. The medical device of claim 1, wherein the end effector comprises a first jaw and a second jaw and when the end effector is in an opened position, the first jaw is spaced from the second jaw and when the end effector is in a closed position, the first jaw overlaps the second jaw.

6. The medical device of claim 1, wherein:
the end effector defines a fluid outlet path, and
when the end effector is in an opened position, the fluid outlet path is in fluid communication with the fluid channel and when the end effector is in a closed position, the fluid outlet path is disconnected from fluid communication with the fluid channel.

7. The medical device of claim 1, wherein the control shaft is electrically conductive and configured to provide electrical power to the end effector.

8. The medical device of claim 1, wherein the control shaft is electrically insulated from the fluid channel.

9. The medical device of claim 1, further comprising:
a post coupled with the control shaft and electrically coupled with the end effector, and
wherein the post defines a first post channel of the fluid channel and a second post channel of the fluid channel.

10. The medical device of claim 9, wherein:
the end effector defines a first fluid outlet path and a second fluid outlet path, and
when the end effector is in an opened position, the first fluid outlet path is in fluid communication with the first post channel and the second fluid outlet path is in fluid communication with the second post channel.

11. A medical device comprising:
a handle, the handle comprising a fluid port, an electrical connector, and an actuator;
an elongate shaft extending from the handle; and
an end effector at a distal end of the elongate shaft, the end effector is in fluid communication with the fluid port and is in electrical communication with the electrical connector, and
wherein actuation of the actuator causes the end effector to adjust position.

12. The medical device of claim 11, wherein the actuation of the actuator to a first position is configured to permit fluid from the fluid port to flow through the end effector and actuation of the actuator to a second position is configured to prevent fluid flow through the end effector.

13. The medical device of claim 11, further comprising:
a fluid channel in fluid communication with the fluid port and extending through the elongate shaft to the end effector.

14. The medical device of claim 11, further comprising:
a control shaft extending through the elongate shaft, the control shaft is coupled with the end effector and the electrical connector.

15. The medical device of claim 11, wherein the electrical connector is electrically isolated from the fluid port.

16. A method comprising:
delivering a cutting tool to a target site within a patient, where the cutting tool is located at distal end of an elongate shaft;
delivering fluid to the target site through the elongate shaft of the cutting tool;
applying electrical power to the cutting tool; and
cutting tissue with the cutting tool using the electrical power.

17. The method of claim 16, wherein the fluid is delivered to the cutting tool and the electrical power is applied to the cutting tool simultaneously.

18. The method of claim 16, wherein a control shaft is coupled with an actuator and the cutting tool and the method further comprises actuating the actuator from a first position in which the cutting tool is a first orientation configured to allow fluid to pass through the cutting tool to a second position in which the cutting tool is in a second orientation configured to prevent fluid from passing through the cutting tool.

19. The method of claim 16, wherein cutting tissue with the cutting tool comprises:
spacing a first jaw of the cutting tool from a second jaw of the cutting tool;
positioning the first jaw and the second jaw around tissue; and
engaging tissue with the first jaw and the second jaw.

20. The method of claim 19, further comprising:
delivering fluid from the cutting tool while the first jaw is spaced from the second jaw.
